(19) United States
(12) Patent Application Publication
(10) Pub. No.: US 2026/0157704 A1
(43) Pub. Date: Jun. 11, 2026

(54) NON-INVASIVE HEALTH MONITORING SYSTEM

(71) Applicant: Abu Dhabi University, Abu Dhabi (AE)

(72) Inventors: Murad M Al-Rajab, Abu Dhabi (AE); Kamel Ahmed Qassem, Abu Dhabi (AE); Sara Mahmoud Seyam, Abu Dhabi (AE); Youssef Salem Al-Hamadi, Abu Dhabi (AE); Ahmed Hadi Al-Dubaisi, Abu Dhabi (AE)

(73) Assignee: Abu Dhabi University, Abu Dhabi (AE)

(21) Appl. No.: 18/972,682

(22) Filed: Dec. 6, 2024

(51) Int. Cl. A61B 5/00; G16H 40/63

(52) U.S. Cl. CPC A61B 5/742; A61B 5/0022; G16H 40/63

(57) ABSTRACT

A method includes receiving electronic information about a person's vital signs. The electronic information is not received from any wearable device on the person. The method includes generating displayable information that includes the person's vital signs. The displayable information is shown in real-time and the displayable information is encrypted. The method includes displaying the displayable information.

### BACKGROUND

The healthcare sector places a high priority on the accurate and timely monitoring of vital signs as these are crucial for effective medical intervention. Despite advancements, current health monitoring systems face several limitations. For example, in the Intensive Care Unit (ICU), patients often experience significant discomfort. This discomfort primarily stems from the multitude of cables and tubes attached to the patient, which can intensify pain levels, especially for individuals coping with severe burns or other acute conditions. Also, caring for infants in the Intensive Care Nursery (ICN) presents its own set of challenges. Premature babies, known for their restlessness, heighten the probability of false alarms through frequent disconnections of monitoring wires, consequently placing additional stress on medical staff and caregivers.

The inability of existing systems to provide non-intrusive, continuous monitoring outside hospital settings may lead to delayed detection of critical health incidents and consequently poorer health outcomes. There is presently no system that provides for non-intrusive and continuous monitoring of a person's vital signs.

### BRIEF DESCRIPTION OF DRAWINGS

FIG. 1 is a diagram of an example process;

FIG. 2 is a diagram of an example process;

FIGS. 3A, 3B, and 3C are diagrams of an example process;

FIG. 4 is a diagram of an example process;

FIG. 5 is a diagram of an example process;

FIG. 6 is a diagram of an example graph;

FIG. 7 is a diagram of an example graph;

FIG. 8 is a diagram of an example environment;

FIG. 9 is a diagram of an example environment;

FIG. 10 is a diagram of an example environment;

FIG. 11 is a diagram of an example environment;

FIG. 12 is a diagram of an example screenshot;

FIG. 13 is a diagram of an example screenshot;

FIG. 14 is a diagram of an example screenshot;

FIGS. 15-25 are diagrams of example screenshots;

FIGS. 26-28 is diagrams of example graphs;

FIG. 29 is a networking environment; and

FIG. 30 is a diagram of a computer.

### DETAILED DESCRIPTION OF PREFERRED EMBODIMENTS

The following detailed description refers to the accompanying drawings. The same reference numbers in different drawings may identify the same or similar elements.

Systems, devices, and/or methods described herein an innovative non-invasive remote healthcare monitoring system that utilizes Wi-Fi Channel State Information (CSI) and artificial intelligence (AI) algorithms, including Convolutional Neural Networks (CNN), to monitor heart and respiratory rates accurately without wearable devices. In embodiment, the systems, device, and/or methods described herein are for a Vi-Fi system. In embodiments, a Vi-Fi system can include an electronic application that provides real-time monitoring, a secure web dashboard for healthcare providers, and data privacy through robust encryption.

In embodiments, the systems, devices, and/or methods provide for high accuracy in estimating breathing and heart rates, and significantly reduces false alarms. For example, the systems, devices, and/or methods described herein can achieve 95% precision in breathing rate estimation, 92% in heart rate estimation, and enhances patient comfort and compliance. In embodiment, a Vi-Fi system (as described herein) provides for a secure, efficient solution for vital signs monitoring by improving patient outcomes and reducing healthcare costs through accessible and effective monitoring technology.

In embodiments, systems, devices, and/or methods described herein combine Wi-Fi CSI and AI algorithms to create a smart, secure, and non-invasive health monitoring system. In embodiments, the system can detect macro-scale body movements and vital signs like heart rate and respiration rate without the need for physical contact. Accordingly, this innovative approach addresses the key problems identified in current systems, offering a more comfortable, reliable, and continuous monitoring solution. In embodiments, Vi-Fi's significance lies in its potential to transform healthcare by providing a more efficient and patient-friendly monitoring environment. In embodiments, the system's ability to offer real-time alerts and continuous tracking can significantly improve patient care, particularly for vulnerable populations. Moreover, by reducing the dependency on traditional, invasive monitoring methods, Vi-Fi can enhance patient comfort and compliance, leading to better health outcomes and reduced healthcare costs.

According, the systems, devices, and/or methods (a) combines Wi-Fi Channel State Information (CSI) with AI algorithms to accurately monitor heart and respiratory rates without the need for wearable devices or physical contact; (b) implementation of a user-friendly electronic application that allows patients and caregivers to monitor vital signs in real-time, receive alerts, and communicate directly with healthcare providers; (c) development of a comprehensive electronic dashboard for healthcare providers, enabling secure, real-time access to patient data, visualization of health metrics, and efficient management of patient care; (d) encryption methods for secure data transmission between the monitoring devices, mobile application, and web dashboard, ensuring patient data privacy and compliance with healthcare regulations; and extensive testing and validation of the Vi-Fi system in real-world scenarios, reducing false alarms and improving the accuracy of vital sign monitoring.

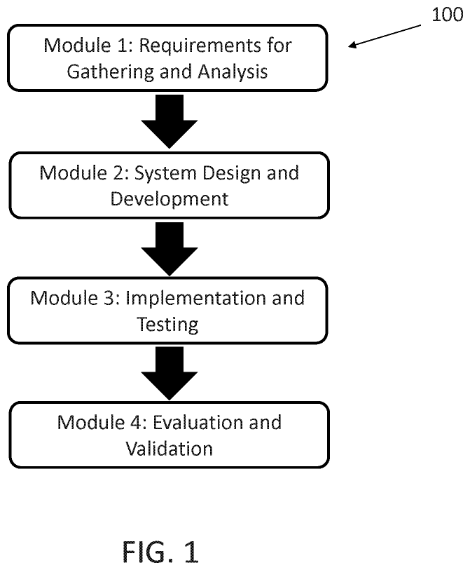

FIG. 1 describes an example overview of a process 100 for obtaining vital sign information by the systems, devices, and/or methods described herein. As shown in FIG. 1, at module 100, requirements are determined for obtaining information. In embodiments, obtaining information about the types of users for the system (e.g., hospitals, nursing homes, etc.), the computing and software systems used, and the analysis of information obtained from user interviews. At module 200, the architectural design is determined, development of AI algorithms for CSI data processing, implementation of an electronic application (which includes a user-interface dashboard), and an integration of security systems. At module 300, the system is tested which includes data preprocessing, training of the AI algorithms, and security testing. At module 400, evaluation of different electronic and computing features is conducted and includes evaluation of the accuracy of the vital sign information, determining the usability of the user-interface dashboard, and analysis of the electronic application.

In embodiments, the monitoring system effectively uses Wi-Fi CSI to precisely estimate heart rate and respiration rate. By combining the power of the CSI and AI, the monitoring system is a smart and secure contactless system to monitor vital signs in real-time. Accordingly, this system can be used in assisting the prevention of potential issues from heart attacks, breathing problems, and other health issues.

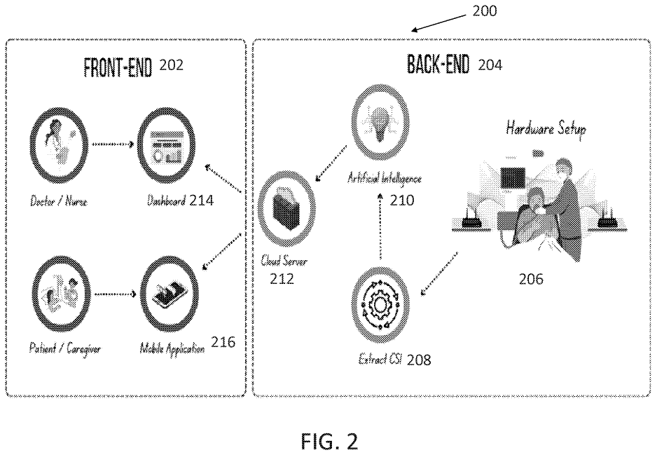

FIG. 2 describes example system 200 that provides an overview of a monitoring system for monitoring vital signs. As shown in FIG. 2, front-end 202 and back-end 204 are shown. As shown in FIG. 2, extract CSI information 208 is received based on hardware set up 206. In embodiment, hardware set up 206 includes setting up routers and other devices in a hospital, nursing home, residential home, etc., to obtain CSI information. As shown in FIG. 2, AI system 210 analyzes the CSI data to extract heart and respiration rates and identify abnormalities. In embodiments, the analyzed CSI data is then generated into a user-friendly format which is electronically displayed on web dashboard 214 via a server (such as cloud server 212) that is accessible to healthcare professionals. To safeguard patient privacy and adhere to global medical data standards, robust access controls are implemented, following protocols like HIPAA (Health Insurance Portability and Accountability Act).

Additionally, the system 200 can incorporate an electronic application (such as a mobile application 216) for patients and caregivers, enabling continuous tracking of vital signs and communication with the medical team from anywhere. In embodiments, web dashboard 214 website and mobile application 216 may use security principles outlined by OWASP (Open Web Application Security Project) and NIST (National Institute of Standards and Technology) standards. Furthermore, encryption is employed to secure data both in transit and in storage. In embodiments, system 200 may also be used for remote monitoring of individuals' health from the comfort of their homes.

In embodiments, a monitoring system (of vital signs, also known as the Vi-Fi system) as described in the example figures, has different functional requirements that focus on vital sign monitoring, data processing, user interaction, and security. In embodiments, the Vi-Fi system uses Wi-Fi signals to monitor the patient's vital signs without requiring physical contact with the person for whom the monitoring is being conducted upon. In embodiments, the Vi-Fi system can continuously capture and process Wi-Fi CSI data to estimate heart and respiration rates in real-time.

In embodiments, the Vi-Fi system can provide a clear representation of the patient's vital signs (e.g. via graphics and text) through both the web and mobile application interfaces. In embodiments, the Vi-Fi system uses AI algorithm to process the CSI data, estimate and monitor the patient's vital signs. In embodiments, the Vi-Fi system automatically generate and send alerts to a medical team with the patient is in a critical condition. In embodiments, the Vi-Fi system helps a medical team, patients, and caregivers to communicate using the web and mobile applications. In embodiments, the Vi-Fi system allows patients to monitor their health from their homes, and keep the caregiver updated on their loves one's health.

In embodiments, the Vi-Fi system allows a medical team to access and check the patients' medical history records for trend analysis and long-term monitoring. In embodiments, the Vi-Fi system encrypt all the data transmitted between the router, server, and User interfaces using methods such as AES-256. In embodiments, the Vi-Fi system supports multiple user roles such as doctors, nurses, and patients with correct access controls. In embodiments, the Vi-Fi system must be accessible via both web-browsers and mobile devices. In embodiments, the Vi-Fi system should be operational 24/7

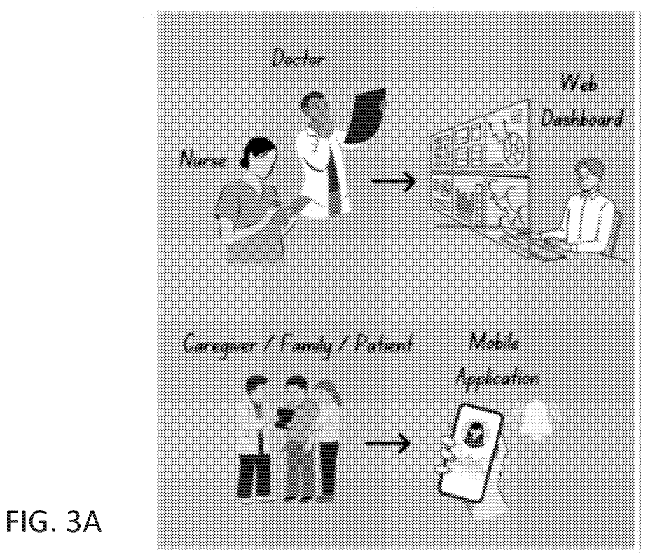

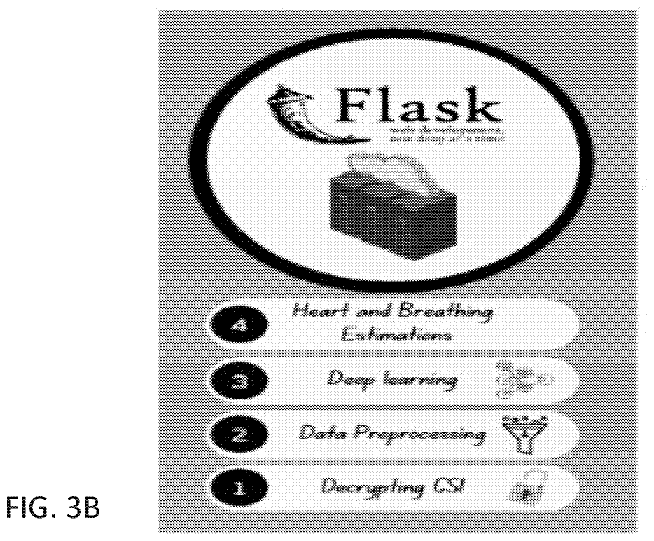

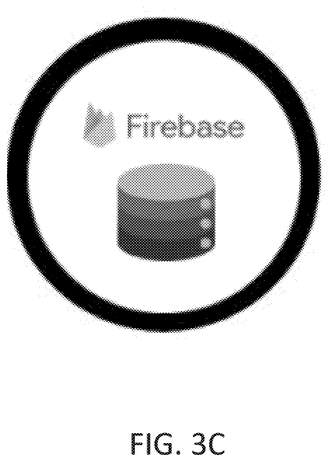

FIGS. 3A, 3B, and 3C describe an example three-tier architecture. As shown in FIG. 3A, a presentation tier includes the web and mobile interfaces that facilitate user interactions. As shown in FIG. 3B, an application tier involves backend services responsible for receiving and decrypting CSI signals, vital sign monitoring, data preprocessing, and AI-based analysis. In embodiments, the application tier manages data exchange among users and runs the neural network algorithm for monitoring heart and respiration rates, triggering alerts for irregular readings. Additionally, the application layer encompasses the server, which acts as an intermediary between the presentation tier and the data tier. In embodiments, the server may be programmed using Python, specifically the socket.io library.

As shown in FIG. 3C, the data tier includes the database responsible for managing data. The data tier includes one or more computing device that store all patient and healthcare information that may have information displayed on an electronic application and/or a web-based browser. In embodiments, system 200 may use two TL-WDR4300 routers, two laptops (Dell Inspiron 7548 laptop and an HP Victus Gaming Laptop 15-falxxx), Ethernet Cat 5E/6 cables, and a WHOOP Strap. In embodiments, the router adheres to the 802.11 standards and provides dual-band connectivity, operating on 2.4 GHz and 5 GHz simultaneously, with transmission rates of 300 Mbps and 450 Mbps. In embodiments, the router may be equipped with three detachable omnidirectional antennas that radiate signals in all directions. The router includes the Atheros 9580 chipset, which is dedicated to CSI extraction and is compatible with the Atheros CSI-Toolkit, facilitating detailed wireless analysis. In embodiments, a strap may be designed to be worn on the wrist and syncs wirelessly with a mobile application, offering real-time health data tracking.

In embodiments, a receiving router (Rx) captures important data signals, including the CSI, from a nearby transmitting router (Tx). To safeguard the confidentiality of the patient's data, the CSI data collected at the Rx end undergoes encryption using the AES256 algorithm before being transmitted to the server. In embodiments, once received by the server, this encrypted CSI data is decrypted for further processing. Following that, primitive processing is applied to the CSI data since the CSI values obtained from standard Wi-Fi devices inherently contain noise. In embodiments, this noise arises from hardware imperfections such as carrier frequency offsets, changes in transmission power, and environmental variations, including path loss, shadowing, and fading. Thus, to enhance the accuracy of our system, the CSI streams will undergo primitive signal processing.

In embodiments, the CSI values can be represented as a complex number. A complex number is represented with two constants as shown in equation (1), where a represents the real value of the complex number and b represents the imaginary value of the complex number. In embodiments, each CSI value consists of both an amplitude and phase component of the signals received by the wireless receiver. Therefore, prior to preprocessing our CSI data, the CSI data is segmented into amplitude and phase data. In embodiments, the AI model (Deep Learning Algorithm) accept real values as input such as amplitude and phase. In embodiments, raw CSI is received in the form of a complex number and generally complex number contain two parts, real and imaginary, and expressed as: a+bi where “a” is constant and represent the real part of the complex number, and “b” also a constant that represent the imaginary part of the complex number. Thus, in embodiments, amplitude and phase information is extracted from complex numbers. In embodiments, a NumPy library provides functions such as abs( ) to compute the amplitude (magnitude) and angle( ) to determine the phase angle. By applying these functions to a given complex number (a+bj), amplitude and phase angle is extracted.

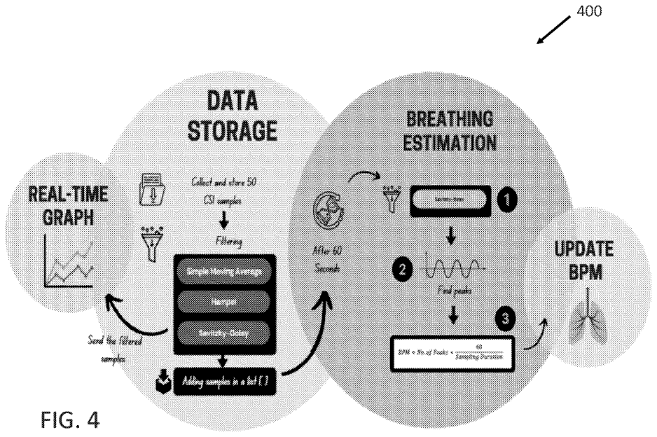

FIG. 4 describes an example 400 process estimates the breathing rate based on filtering techniques and also using electronic information (such as peaks) to identify the breathing rate. In a non-limiting example, 50 CSI amplitude samples are obtained (continuously) and is applied with three filters: Simple Moving Average, Hampel, and Savitzky-Golay. In embodiments, the Simple Moving Average filter helps smoothen out the data by averaging out short term fluctuations reducing the impact of random noise that could interfere with the accurate peak detection. In embodiments, the Hampel filter further cleans the data by identifying and removing outliers (sudden spikes or drops in data), which can distort the analysis. In embodiments, the Savitzky-Golay filter is applied twice with different window sizes to further refine the data. Initially, it helps to smooth the data without distorting the signal, maintaining the integrity of the breathing cycles.

In embodiments, the received data is smoothed over a longer period which allows for a more precise peak detection for calculating the breathing rate. In embodiments, the filtered data, before applying the second Savitzky-Golay filter, is transmitted to the front end of the medical team's web dashboard for real-time visualization and stored for further processing. After a particular amount of time (e.g., 40 seconds, 60 seconds, etc.), the data is reprocessed using the second Savitzky-Golay filter. Finally, once peaks are identified, the breathing rate is calculated using the equation (2) which determines the detected breathing cycles within the last minute based on the number of peaks identified. Equation (2) is:

$$Breathing Rate = number of peaks \times ( \frac{60}{s a m pling duration} ) \qquad ( 2 )$$

In a non-limiting example, for the heart rate implementation, after collecting 500 packets, subcarrier selection is performed by choosing 16 subcarriers with the highest variance out of the 56 available subcarriers. In embodiments, subcarriers are individual frequency components within the overall Wi-Fi channel bandwidth used to transfer data efficiently through the network, each carrying a portion of the transmitted data such as portion of a packet. In embodiments, the packet refers to the data being transmitted. In embodiments, the use of multiple subcarriers allows for more robust data transmission and comprehensive sensing of the patient's environment. In embodiments, the selection process focuses on the subcarriers with the most variation, meaning the subcarriers that changed the most, assuming the breathing and heart rate are part of that motion as not all 56 subcarriers pass through the patient. By selecting 16 subcarriers, this also reduces the computational power required for the AI, as only 16 subcarriers from each packet are processed instead of 56.

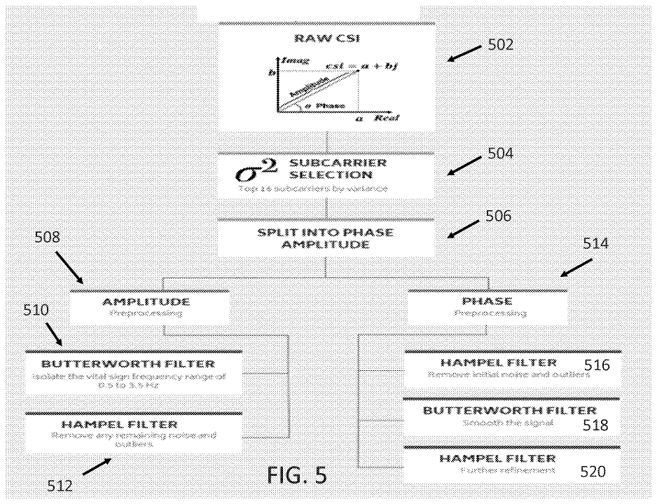

In embodiments, the selected CSI data undergoes preprocessing to reduce noise and outliers as shown in FIG. 5. As shown in FIG. 5, at step 502, raw CSI data is received and at step 504, 16 subcarriers are selected. At step 506, the phase and amplitude components are split and preprocess them separately processed. Thus, at step 508, the amplitude is processed; and, at step 514, the phase is processed.

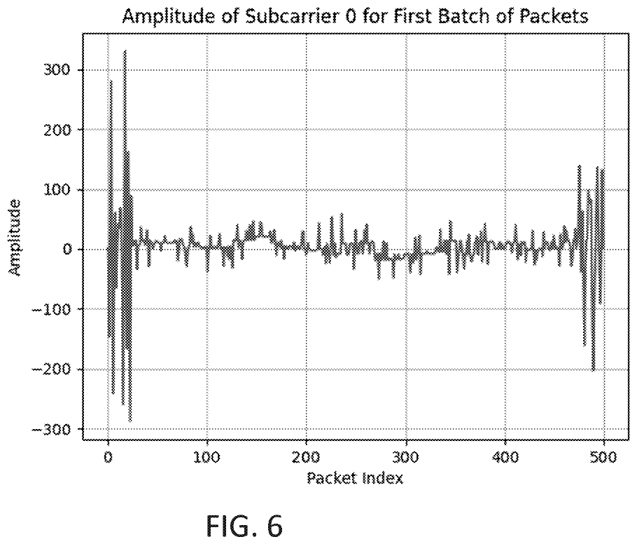

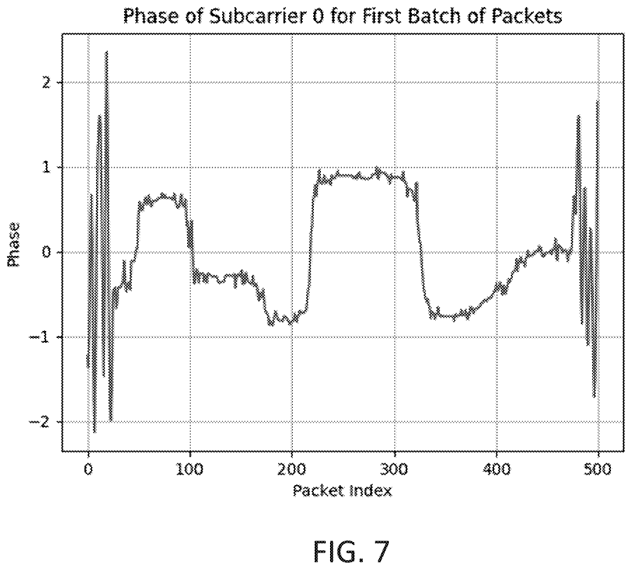

For the amplitude data, at step 510, a Butterworth filter isolates the vital sign frequency range of 0.5 to 3.5 Hz, followed by, at step 512, a Hampel filter is applied to remove any remaining noise and outliers. At step 516, the phase data undergoes a similar process, starting with a Hampel filter to remove noise and outliers, followed by, at step 518, a Butterworth filter to smooth the signal, and at step 520 with a second Hampel filter pass for further refinement. The results of a subcarrier after filtration are shown in FIGS. 6 and 7. As shown in FIG. 6, this represents the amplitude component after preprocessing. As shown in FIG. 7, this illustrates the phase component after applying the filters.

In embodiments, once the phase and amplitude data are filtered, the filtered phase and amplitude data are combined into a 3D matrix (packets×subcarriers×components) and reshaped into a 4D tensor. In embodiments, the extra dimension represents the batch size for processing multiple samples simultaneously. In embodiments, the developed Convolutional neural network (CNN) consists of convolutional blocks followed by max pooling layers, batch normalization, and fully connected layers for feature extraction and prediction. In embodiments, the final layer is the output layer where the heart rate is determined. Finally, these rates will be transmitted to our server, enabling continuous real-time monitoring of the results. In embodiments, the data is synchronized with the web portal and electronic mobile application for user access and interaction.

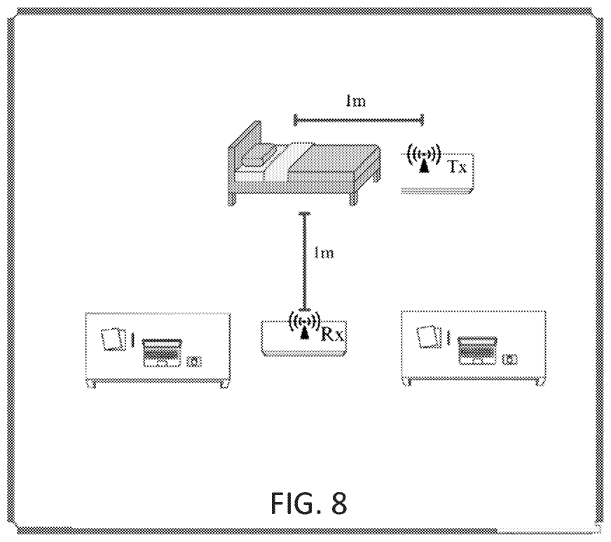

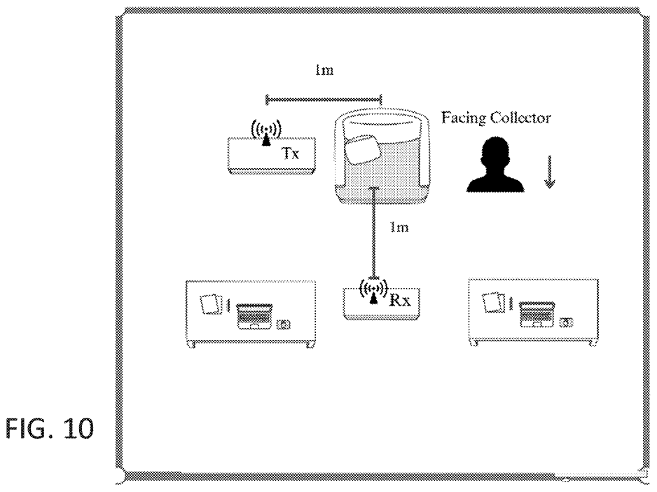

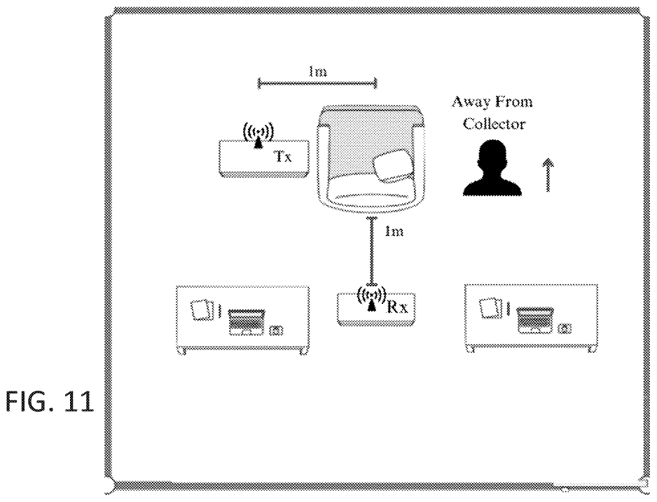

In a non-limiting example, a quiet room with interior dimensions of 4.0 by 3.2 by 2.9 meters (length, width, height) may be used and is shown in FIG. 8. In this non-limiting example, a Rx router is placed on the side of the bed and the Tx router is placed at the end of the bed, forming a right angle. This setup mimics the same environmental conditions as the “eHealth CSI” dataset, which we used to train our AI model for heart rate estimation. In this non-limiting example, the patient faces different directions to investigate how patient positions might affect system performance. Alternatively, the patient can also face the receiver (FIG. 10) or face away from it (FIG. 11) or be in a sleeping position.

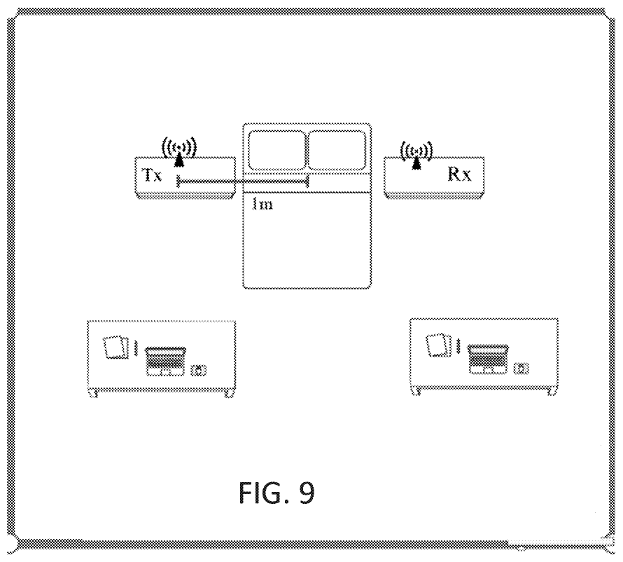

FIG. 9 displays a secondary, non-limiting example, setup. In this setup, the patient is lying on a bed with the routers placed on both sides, directed toward the patient's chest, and separated by 1 meter. This alignment ensured a clear line of sight for optimal signal transmission and reception. In all cases, the routers were positioned 1 meter apart. The secondary setup was included because it seemed more feasible and convenient for hospital implementation.

In embodiments, the AI architecture performance may be assessed using key metrics like loss and Mean Absolute Error (MAE), which measure accuracy in predicting heart rates from CSI data. Accordingly, the AI system can estimate heart rates in real-world settings, achieved through training and evaluation procedures. Additionally, a dataset may be generated that includes data for both respiration and heart rate. In embodiments, the dataset can be used to train an AI model to simultaneously estimate both these vital signs leveraging their intrinsic relationship.

In embodiments, an encryption process may be used to secure the CSI data. In embodiments, once the receiver router obtains the CSI data from the transmitter, the receiver router encrypts the data before sending it to the server. In embodiments, the encryption process uses AES-256 to encipher the CSI data, ensuring confidentiality and integrity during transmission. This implementation focuses on receiving data and encrypting it before transmission over UDP (user datagram protocol) and initializes encryption parameters, such as the key and initialization vector, for AES encryption. Upon receiving data, necessary padding is calculated (e.g., calculated by determining how many bytes are needed to make the data length a multiple of the block size and appended to ensure the data length aligns with the encryption block size, which is 16 bytes for AES256). Using AES CBC encryption with the provided key and initialization vector, the data is encrypted and then sent over UDP, ensuring secure transmission over the network

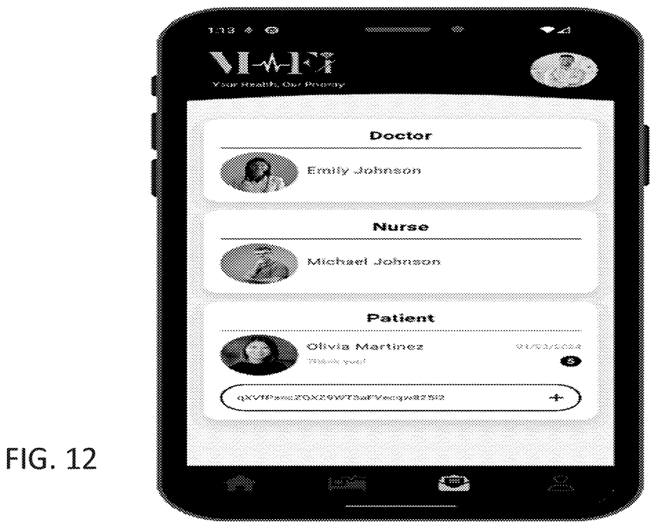

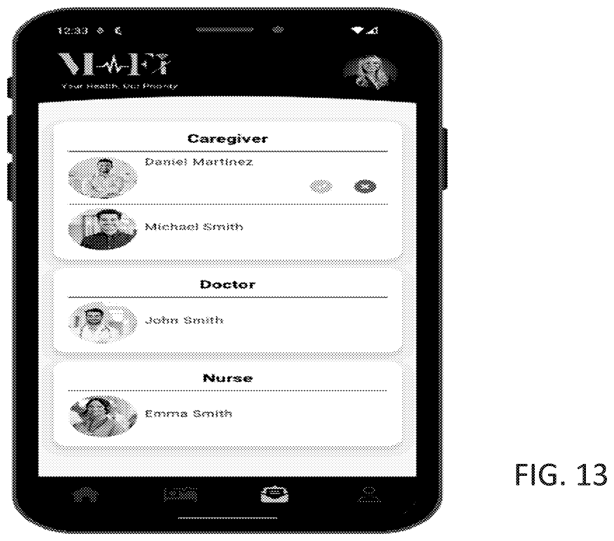

In embodiments, access control mechanisms allow only authorized users to view patient data which include Role Based Access Control (RBAC) and Discretionary Based Access Control (DBAC), giving the patient full control over their data. RBAC ensures that users are directed to appropriate parts of the web/mobile application based on their roles, maintaining security and access control throughout the application. For instance, if the user is an administrator, they are redirected to the admin dashboard. If the user belongs to the medical team and their specialization is ‘Doctor’, they are redirected to the doctor dashboard with their user data. Next, DBAC enables the patient to grant or revoke access from the caregiver. FIG. 12 shows the caregiver sending access request to the patient by adding his ID, and FIG. 13 shows that the patient has the option to accept or reject the access request.

To ensure the security of the system, vulnerability assessments may be conducted using a vulnerabilities repository. In embodiments, these assessments are crucial for identifying and mitigating new weaknesses that could severely impact the functionality of the system.

In embodiments, a request is made from the presentation tier, it is sent to the application tier, which contacts the data tier to retrieve the required data and then responds to the presentation tier. This architecture offers several advantages, such as scalability by allowing independent scaling of each tier based on demand, optimizing resource utilization. In embodiments, the server-side component incorporates robust security measures to protect sensitive data and enhances user-friendliness and responsiveness by streamlining client logic.

In embodiments, the user interaction flow is designed to be intuitive, allowing healthcare professionals to monitor patient data efficiently. Backend services process and display real-time physiological data on the user interfaces. In addition, CSI data was encrypted using AES-256 before transmission to ensure patient data confidentiality. The data was decrypted on the server for further processing and analysis. Accordingly, the system's modularity facilitates easy modifications without impacting other components and maintains availability. Furthermore, a centralized database ensures data consistency, crucial for the effective analysis of vital sign data. Additionally, having separate but interrelated layers facilitates the integration of external plugins and APIs, enabling interaction with third-party services or other applications.

In embodiments, the benefit of the three-tier architecture includes high performance and reliability, which are not achievable with client-side applications. In client-side architecture, most processing occurs on the client device (e.g., browser or mobile), leading to significant reliance on the client's device capabilities and processing power. In contrast, the three-tier architecture distributes processing more effectively, enhancing overall system performance and reliability.

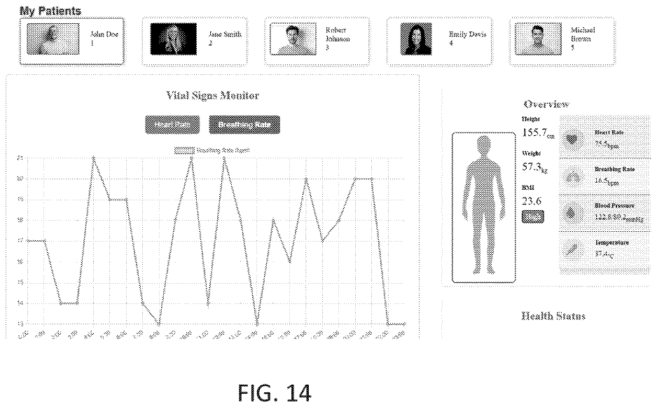

FIG. 14 describes an electronic dashboard. As shown in FIG. 14, a user will see a number of dashboard features. This includes real-time monitoring of vital signs. In embodiments, the system provides real-time monitoring for the medical team. As shown in FIG. 14, a monitoring graph for both heart and breathing rate is provided and also a patient overview section.

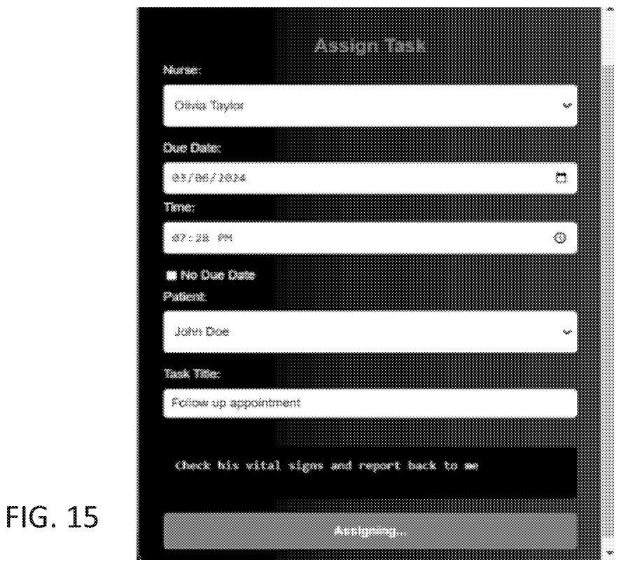

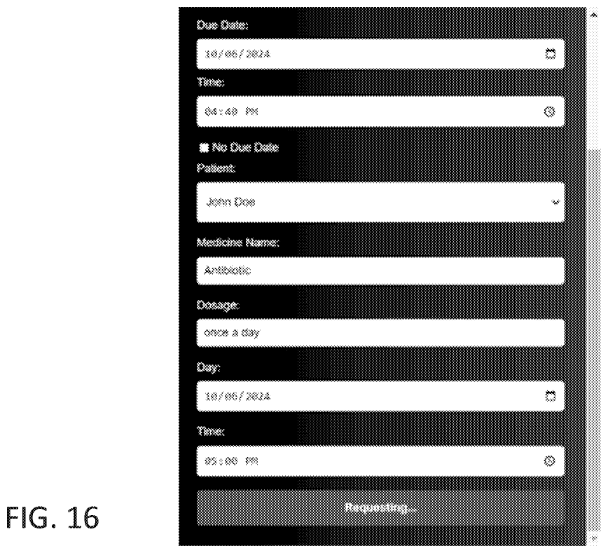

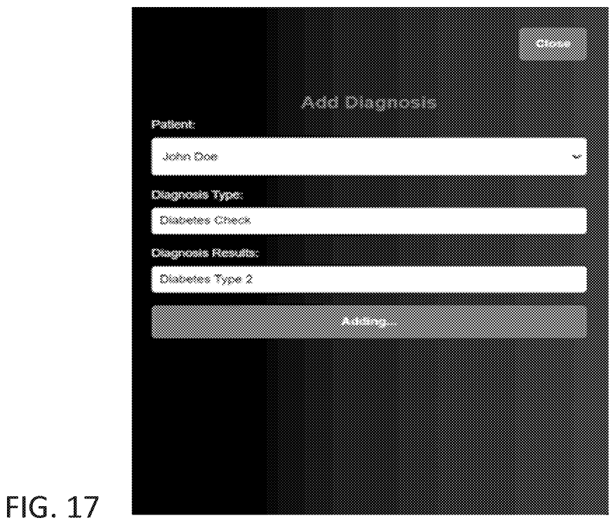

As for the Quick settings shown in the navigation bar, the doctor can assign a task to a nurse as shown in FIG. 15, request a new medication as shown in FIG. 16, and can add new diagnosis as shown in FIG. 17.

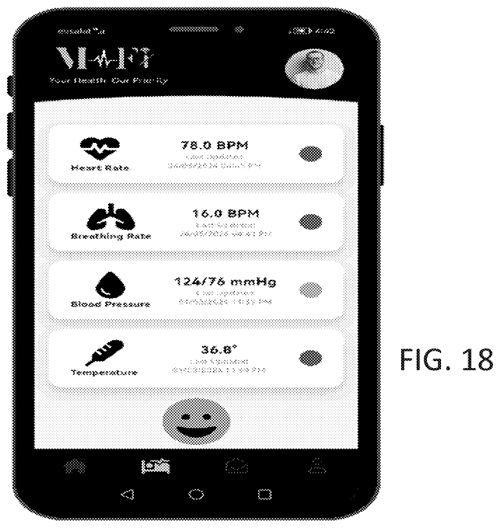

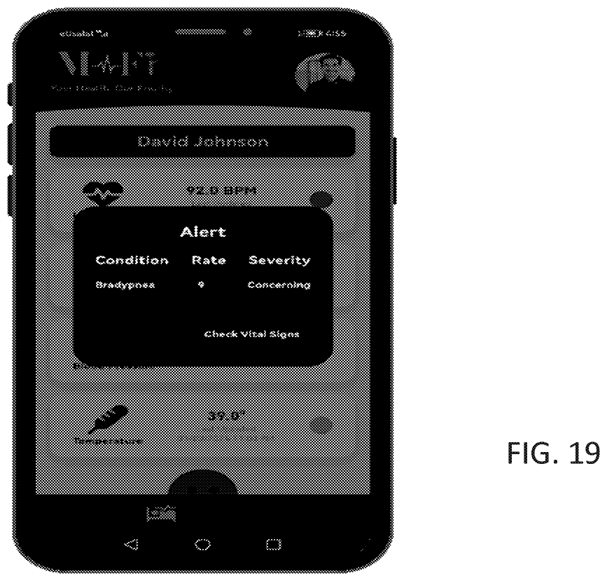

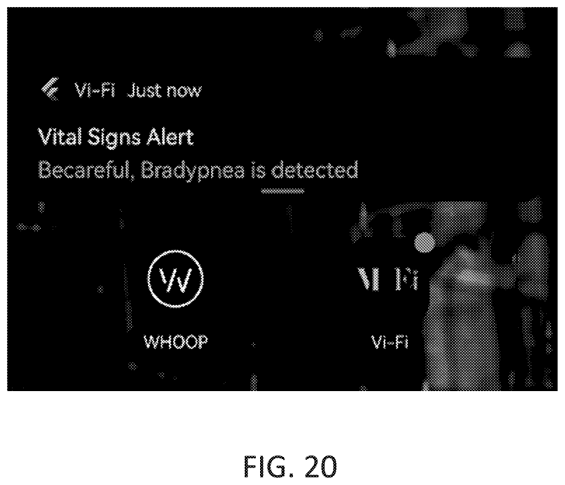

In a non-limiting example, after the patient and caregiver register in our mobile application and login to their dashboard, they will be interfaced with many features to monitor vital signs in real time. As shown in FIG. 18, the patient and caregiver have access to an electronic page dedicated to tracking vital signs, which are updated in real-time, minute by minute. In addition to that, if the vital signs of the patient are abnormal, an emergency alert will appear on the middle screen area and trigger an alert sound to grab the caregiver's attention for any emergencies as shown in FIG. 19. Even if the caregiver is not in the application itself, they will receive an application notification of the alert case as shown in FIG. 20.

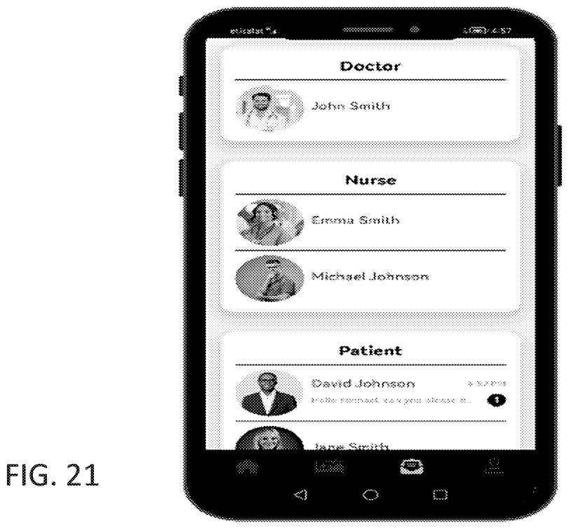

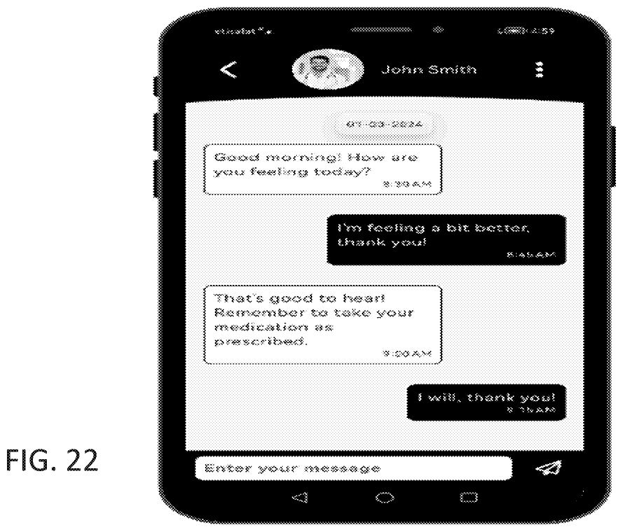

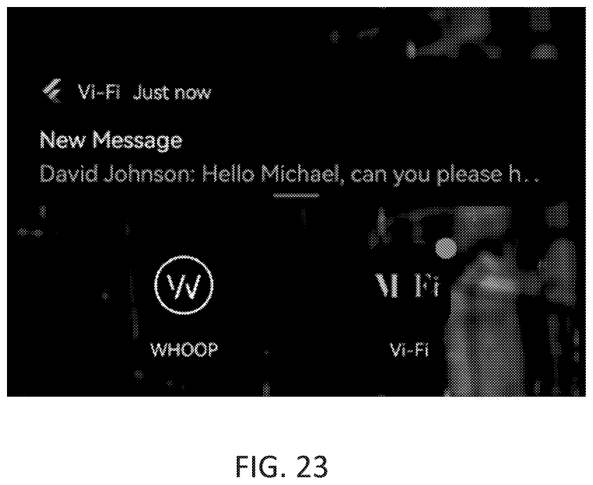

In embodiments, the Vi-Fi system includes a seamless communication bridge between the patient, caregivers, and the medical team. To achieve this seamless communication, an electronic page (i.e., screenshot) is shown in FIG. 21 displays all the user contacts. In embodiments, this page displays the last message the contact sent to the user, along with how many unread messages are available. FIG. 22 shows the messaging screen where the user can directly communicate with any of the other stakeholders in real-time. Like the emergency alerts, if the user is not in the application, they will receive a notification of the new messages received as shown in FIG. 23.

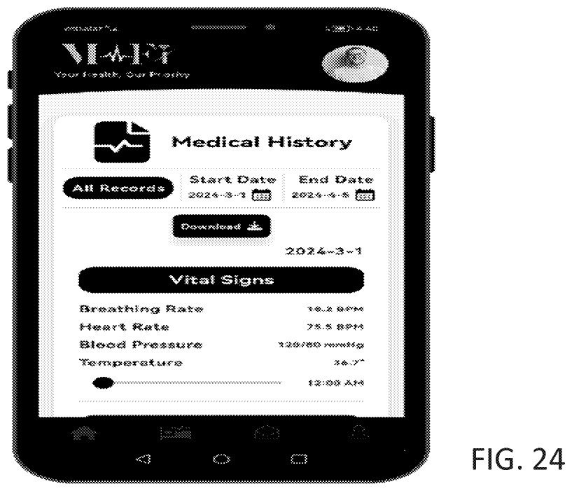

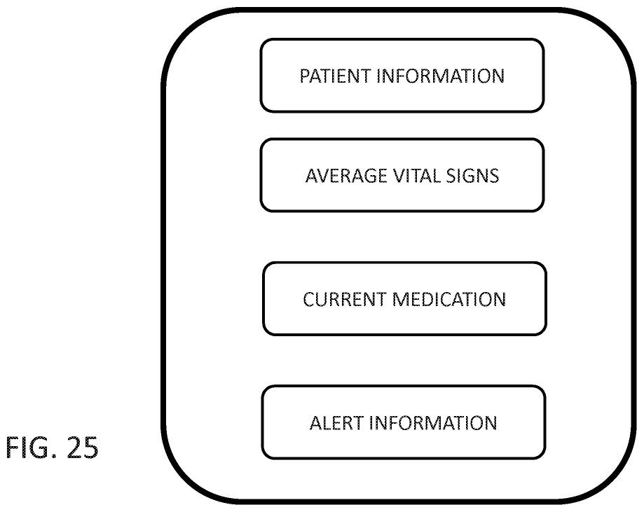

FIG. 24 shows an electronic page that displays the medical history screen, which contains features that allow the patient to easily view the desired data and share them with convenience. The user can filter the results based on a date range by disabling the “All Records” button and entering the start and end date of the reporting period. Below that, there is a “Download” button, which allows the user to download the medical history of the specified period in PDF format as shown in FIG. 25. In addition, there is a slider in the vital signs section, which allows the patient to view the hourly average vital signs of the whole day in a user-friendly manner.

In embodiments, the Vi-Fi system aims to enhance patient care by providing a smart solution to monitor patient vital signs non-invasively utilizing wireless Wi-Fi signals. Furthermore, the Vi-Fi system offers a user-friendly platform for the medical team to ease patient monitoring with visualization and enhance communication between doctors and nurses. Additionally, the Vi-Fi system gives patients full access and control over their health records and allows caregivers to easily monitor their loved ones remotely. To meet our goals, we developed our own AI model to estimate heart rate, created a new processing method to estimate breathing rate, and developed a web dashboard for the medical team and a mobile application for the patients and caregivers.

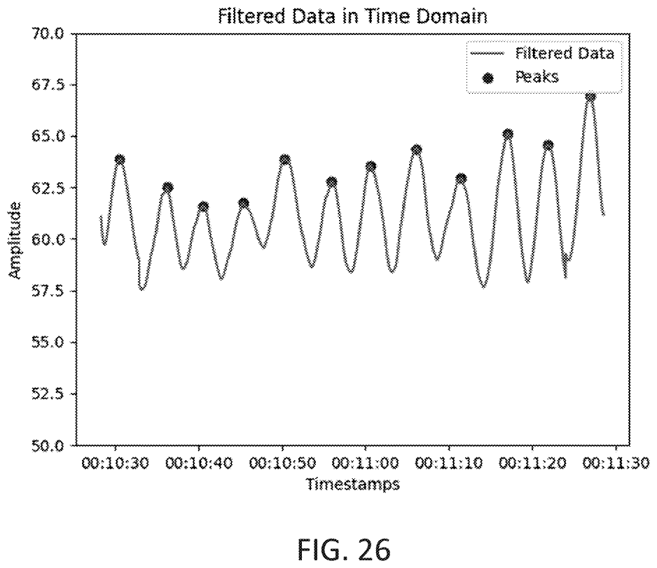

FIG. 26 shows an electronic graph that can be sued for breathing rate estimation. In embodiments, filtering techniques can be used the number of peaks to identify the breathing rate. Using our unique signal processing methods, we were able to accurately identify the inhalation and exhalation phases of the patient. Based on the filtering techniques, it can be determined from FIG. 26 that a patient took 12 breaths within the minute shown in the figure. In embodiments, the estimated error margin is expected to be 1 bpm as data is collected for 60 seconds (1 full period) and relying on counting the peaks, which are integers.

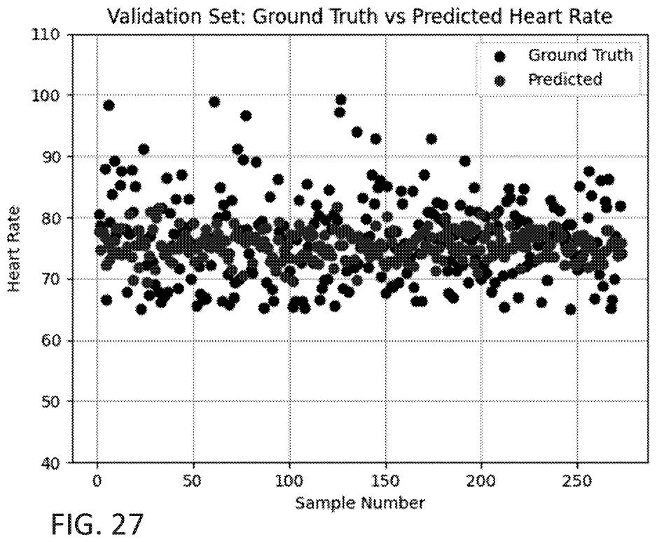

In embodiments, the CNN algorithm developed for estimating heart rate from CSI data has yielded impressive results, achieving an average Mean Absolute Error (MAE) within the range of 5-6 beats per minute (bpm). In embodiments, the systematic preprocessing steps involving the removal of outliers and the extraction of heart rate-related ranges from both the amplitude and phase components. In embodiments, the algorithm ensures both accuracy and reliability in its predictions. FIG. 27 shows the performance of the Vi-Fi system for estimating heart rate.

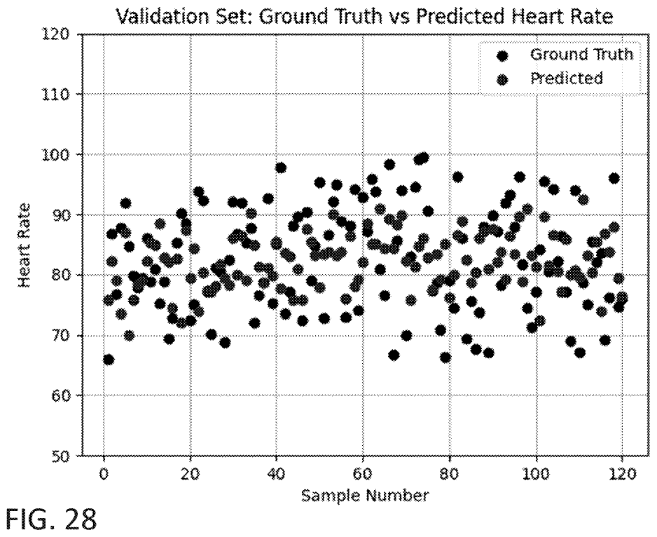

To further validate the robustness and generalization capability of our proposed system, a 10-fold cross-validation is performed on the collected dataset. In embodiments, this involves randomly partitioning the data into 10 equal-sized subsets, using 9 subsets for training and the remaining subset for testing, and repeating this process 10 times with each subset serving as the test set once. The results of this evaluation achieved a MAE of 8 bpm in estimating heart rates. FIG. 28 shows the 10-fold results.

In embodiments, the Vi-Fi system offers heart rate monitoring and employs AI algorithms for enhanced monitoring capabilities. It provides real-time visualization through an interactive web portal and a user-friendly mobile application, improving communication between healthcare providers, patients, and caregivers. In embodiments, the Vi-Fi system focuses on robust vital sign monitoring, particularly heart and breathing rates, and efficiently processes CSI data to detect irregularities. In embodiments, the Vi-Fi system implements AES-256 encryption and RBAC to secure data and access. In embodiments, the Vi-Fi operates with just two commodity routers, eliminating the need for specialized hardware. In embodiments, the Vi-Fi system also stands out by using advanced AI algorithms to analyze patients' vital signs in real-time.

Furthermore, the Vi-Fi system provides a number of benefits. This includes real-time monitoring and communication features that enable healthcare providers to remotely monitor patients' vital signs, which is a critical component of effective telemedicine practices. This remote monitoring capability enhance patient outcomes and reduces the need for in-person visits which is particularly beneficial for patients in remote areas or those with limited mobility. Environmentally, Vi-Fi's reliance on the already existing Wi-Fi technology which means that there could be a drastic reduction in the need to manufacture specialty medical equipment and hence, reducing the healthcare industry's carbon emission levels which is standing at 4% of the global net emission. From an economical point of view, the Vi-Fi system is also lower cost compared to the conventional monitoring systems.

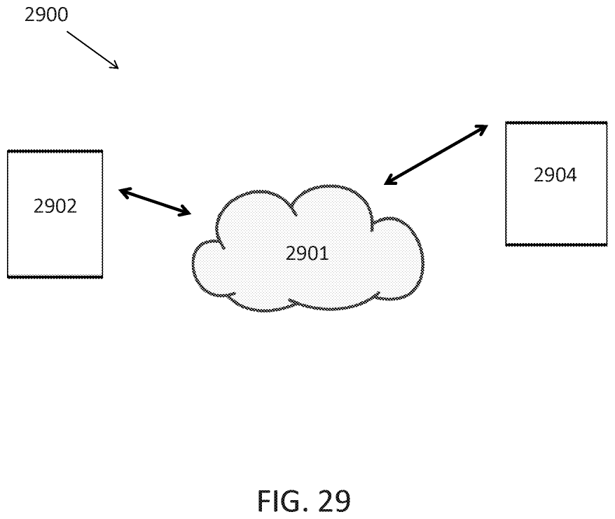

FIG. 29 is a diagram of example environment 2900 in which systems, devices, and/or methods described herein may be implemented. FIG. 29 shows network 2901, user device 2902, user device 2904, and antenna 2906.

Network 2901 may include a local area network (LAN), wide area network (WAN), a metropolitan network (MAN), a telephone network (e.g., the Public Switched Telephone Network (PSTN)), a Wireless Local Area Networking (WLAN), a WiFi, a hotspot, a Light fidelity (LiFi), a Worldwide Interoperability for Microware Access (WiMax), an ad hoc network, an intranet, the Internet, a satellite network, a GPS network, a fiber optic-based network, and/or combination of these or other types of networks. Additionally, or alternatively, network 500 may include a cellular network, a public land mobile network (PLMN), a second generation (2G) network, a third generation (3G) network, a fourth generation (4G) network, a fifth generation (5G) network, and/or another network.

In embodiments, network 2901 may allow for devices describe any of the described figures to electronically communicate (e.g., using emails, electronic signals, URL links, web links, electronic bits, fiber optic signals, wireless signals, wired signals, etc.) with each other so as to send and receive various types of electronic communications.

User device 2902 and/or 2904 may include any computation or communications device that is capable of communicating with a network (e.g., network 2901). For example, user device 2902 and/or user device 2904 may include a radiotelephone, a personal communications system (PCS) terminal (e.g., that may combine a cellular radiotelephone with data processing and data communications capabilities), a personal digital assistant (PDA) (e.g., that can include a radiotelephone, a pager, Internet/intranet access, etc.), a smart phone, a desktop computer, a laptop computer, a tablet computer, a camera, a personal gaming system, a television, a set top box, a digital video recorder (DVR), a digital audio recorder (DUR), a digital watch, a digital glass, or another type of computation or communications device.

User device 2902 and/or 2904 may receive and/or display content. The content may include objects, data, images, audio, video, text, files, and/or links to files accessible via one or more networks. Content may include a media stream, which may refer to a stream of content that includes video content (e.g., a video stream), audio content (e.g., an audio stream), and/or textual content (e.g., a textual stream). In embodiments, an electronic application may use an electronic graphical user interface to display content and/or information via user device 2902 and/or 2904. User device 2902 and/or 2904 may have a touch screen and/or a keyboard that allows a user to electronically interact with an electronic application. In embodiments, a user may swipe, press, or touch user device 2902 and/or 2904 in such a manner that one or more electronic actions will be initiated by user device 2902 and/or 2904 via an electronic application. User device 2902 and/or 2904 may receive electronic information from antenna 2906 and generate and display graphs such as those described in the figures above.

User device 2902 and/or 2904 may include a variety of applications, such as, for example, an e-mail application, a telephone application, a camera application, a video application, a multi-media application, a music player application, a visual voice mail application, a contacts application, a data organizer application, a calendar application, an instant messaging application, a texting application, a web browsing application, a blogging application, and/or other types of applications (e.g., a word processing application, a spreadsheet application, etc.). In embodiments, user device 2902 and/or 2904 may be used to generate graphs (such as those described in FIGS. 6, 7, 26, 27, and 28).

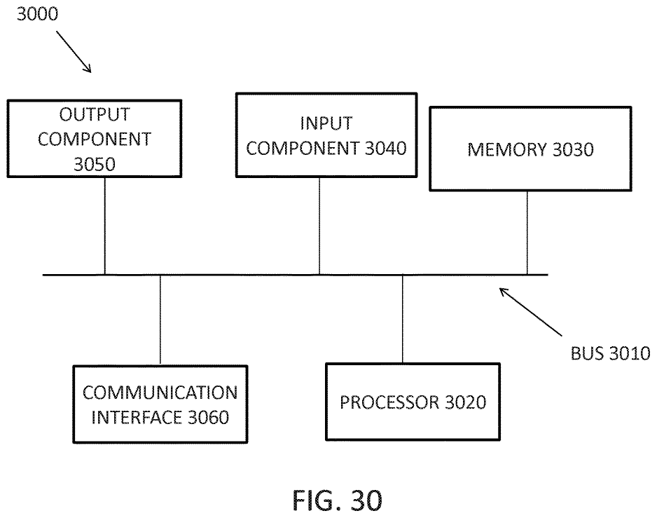

FIG. 30 is a diagram of example components of a device 3000. Device 3000 may correspond to user device 2902, or user device 2904. Alternatively, or additionally, user device 2902 and user device 2904 may include one or more devices 3000 and/or one or more components of device 3000.

As shown in FIG. 30, device 3000 may include a bus 3010, a processor 3020, a memory 3030, an input component 3040, an output component 3050, and a communications interface 3060. In other implementations, device 3000 may contain fewer components, additional components, different components, or differently arranged components than depicted in FIG. 30. Additionally, or alternatively, one or more components of device 3000 may perform one or more tasks described as being performed by one or more other components of device 3000.

Bus 3010 may include a path that permits communications among the components of device 3000. Processor 3020 may include one or more processors, microprocessors, or processing logic (e.g., a field programmable gate array (FPGA) or an application specific integrated circuit (ASIC)) that interprets and executes instructions. Memory 3030 may include any type of dynamic storage device that stores information and instructions, for execution by processor 3020, and/or any type of non-volatile storage device that stores information for use by processor 3020. Input component 3040 may include a mechanism that permits a user to input information to device 3000, such as a keyboard, a keypad, a button, a switch, voice command, etc. Output component 3050 may include a mechanism that outputs information to the user, such as a display, a speaker, one or more light emitting diodes (LEDs), etc.

Communications interface 3060 may include any transceiver-like mechanism that enables device 3000 to communicate with other devices and/or systems. For example, communications interface 3060 may include an Ethernet interface, an optical interface, a coaxial interface, a wireless interface, or the like.

In another implementation, communications interface 3060 may include, for example, a transmitter that may convert baseband signals from processor 3020 to radio frequency (RF) signals and/or a receiver that may convert RF signals to baseband signals. Alternatively, communications interface 3060 may include a transceiver to perform functions of both a transmitter and a receiver of wireless communications (e.g., radio frequency, infrared, visual optics, etc.), wired communications (e.g., conductive wire, twisted pair cable, coaxial cable, transmission line, fiber optic cable, waveguide, etc.), or a combination of wireless and wired communications.

Communications interface 3060 may connect to an antenna assembly (not shown in FIG. 30) for transmission and/or reception of the RF signals. The antenna assembly may include one or more antennas to transmit and/or receive RF signals over the air. The antenna assembly may, for example, receive RF signals from communications interface 3060 and transmit the RF signals over the air, and receive RF signals over the air and provide the RF signals to communications interface 660. In one implementation, for example, communications interface 3060 may communicate with network 2901.

As will be described in detail below, device 3000 may perform certain operations. Device 3000 may perform these operations in response to processor 3020 executing software instructions (e.g., computer program(s)) contained in a computer-readable medium, such as memory 630, a secondary storage device (e.g., hard disk, CD-ROM, etc.), or other forms of RAM or ROM. A computer-readable medium may be defined as a non-transitory memory device. A memory device may include space within a single physical memory device or spread across multiple physical memory devices. The software instructions may be read into memory 630 from another computer-readable medium or from another device. The software instructions contained in memory 3030 may cause processor 3020 to perform processes described herein. Alternatively, hardwired circuitry may be used in place of or in combination with software instructions to implement processes described herein. Thus, implementations described herein are not limited to any specific combination of hardware circuitry and software.

It will be apparent that example aspects, as described above, may be implemented in many different forms of software, firmware, and hardware in the implementations illustrated in the figures. The actual software code or specialized control hardware used to implement these aspects should not be construed as limiting. Thus, the operation and behavior of the aspects were described without reference to the specific software code—it being understood that software and control hardware could be designed to implement the aspects based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While various actions are described as selecting, displaying, transferring, sending, receiving, generating, notifying, and storing, it will be understood that these example actions are occurring within an electronic computing and/or electronic networking environment and may require one or more computing devices, as described in FIG. 29, to complete such actions.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A method, comprising:
receiving, by a computing device, electronic information about a person's vital signs,
wherein the electronic information is not received from any wearable device on the person;
generating, by the computing device, displayable information that includes the person's vital signs,
wherein the displayable information is shown in real-time and the displayable information is encrypted; and
displaying, by the computing device, the displayable information.

2. The method of claim 1, wherein the receiving the electronic information is based on using Wi-Fi Channel State Information (CSI).

3. The method of claim 1, wherein computing device includes a presentation layer, an application layer, and a data layer.

4. The method of claim 1, wherein the person's vital signs are for a heart rate.

5. The method of claim 1, wherein the person's vital signs are for a breathing rate.

6. A device, comprising:
one or more processors to:
receive electronic information about a person's vital signs,
wherein the electronic information is not received from any wearable device on the person;
generate displayable information that includes the person's vital signs,
wherein the displayable information is shown in real-time and the displayable information is encrypted; and
display the displayable information.

7. The device of claim 6, wherein the receiving the electronic information is based on using Wi-Fi Channel State Information (CSI).

8. The device of claim 6, wherein the device is configured to use a presentation layer, an application layer, and a data layer.

9. The device of claim 6, wherein the vital signs are a heart rate or a breathing rate.
